(19) United States
(12) Patent Application Publication
(10) Pub. No.: US 2009/0187118 A1
(43) Pub. Date: Jul. 23, 2009

(54) Multiple Needle System

(76) Inventors: Stanley Kim, Upland, CA (US); Edward Kim, Upland, CA (US); Shyna Kim, Upland, CA (US); Audrey Kim, Upland, CA (US); Esther Kim, Upland, CA (US)

Correspondence Address: Stanley Kim, 421 East 25th Street, Upland, CA, 91784, US

(21) Appl. No.: 12/227,731

(22) PCT Filed: May 29, 2007
(86) PCT No.: PCT/US07/69908

§ 371 Date: Nov. 24, 2008

Related U.S. Application Data
(63) Continuation of application No. 11/420,767, filed on May 29, 2006.
Application No. 11/420,767 is a continuation of application No. 11/594,321, filed on Nov. 7, 2006, now abandoned.
Application No. 11/594,321 is a continuation of application No. 11/602,101, filed on Nov. 20, 2006.
Application No. 11/602,101 is a continuation of application No. 11/637,253, filed on Dec. 11, 2006, now abandoned.

(51) Int. Cl. A61B 5/151; A61B 17/34; A61M 5/32

(52) U.S. Cl. 600/583; 606/181; 604/173

(57) ABSTRACT

A needle system comprising a lancet or syringe having multiple thin needles for accessing bodily fluids so as to inflict less pain and promote faster healing.

The present application claims the benefit of priority from U.S. patent application Ser. No. 11/420,767, filed May 29, 2006, entitled “Painless Blood Sampling Lancet with Bundled Multiple Thin Needles;” U.S. patent application Ser. No. 11/594,321, filed Nov. 7, 2006, entitled “Biopsy device with thin and multiple needles;” U.S. patent application Ser. No. 11/602,101, filed Nov. 20, 2006, entitled “System with a syringe device and a needle device;” and U.S. patent application Ser. No. 11/637,253, filed Dec. 11, 2006, entitled “Hemodialysis needle device having multiple needles mounted on a needle hub.” The entire disclosure of each of the foregoing applications is hereby incorporated by reference.

### BACKGROUND

Blood is subjected to various tests in connection with medical analyses. A widely practiced test is for the determination of glucose levels in blood, in particular in connection with the monitoring and treatment of diabetes. Diabetic patients perform such tests on a frequent basis in order to monitor their blood glucose levels.

In order to draw blood for a blood glucose test, patients typically use a lancet device. Typical lancets have a single needle sized in the range of 21-33 gauge. The lancets are used to puncture the skin of a patient and draw blood for glucose tests.

Each time a lancet pricks the skin of patient, however, the patient experiences pain. Among other considerations, the amount of pain from a lancet corresponds to the size of the wound inflicted by the lancet, as well as the location of the wound. Small lancet wounds may not provide enough blood for a sample, while large wounds may produce considerable pain and heal slowly.

Hollow needle devices having multiple needles have been proposed for use in delivering or withdrawing fluids from an individual. U.S. Pat. No. 7,083,592, for example, provides an apparatus having three needles for delivering therapeutic substances to a patient subcutaneously. U.S. Pat. No. 5,415,182 discloses a multiple needle biopsy instrument for obtaining multiple specimens from a patient. These instruments, however, are not adequate to meet a variety of medical needs for access to patients'bodily fluids.

### SUMMARY

In one embodiment, the present needle system comprises a lancet having multiple needles for obtaining a blood sample from a patient. The lancet employs very thin needles, generally at least 34 gauge in diameter or thinner, which individually may not be able to produce enough blood to perform a test, such as a blood glucose test, but which collectively draw enough blood when used to puncture the skin of a patient. Because the needles are very thin, each needle elicits less pain than a comparably thicker needle when puncturing the skin. The wounds created by such thinner needles also heal more quickly than comparable needles that are wider in diameter.

A needle device comprising one or more needle hubs; a plurality of needles attached to and in fluid communication with the one or more needle hubs, each of the needles being parallel to each other; and a fluid conduit attached at a proximal end to the one or more needle hubs. The fluid conduit can be either a syringe or a catheter, such as a catheter attached to a hemodialysis machine. A syringe device according to this embodiment can comprise a plurality of connecting tips, each of which is connected to a needle hub, in which case each needle hub can comprise a single needle, although multiple-needle hubs are also contemplated. A syringe having a single connecting tip can also be used if the needle hub retains a plurality of needles.

In embodiments in which the present needle system is used for arteriovenous access for hemodialysis, the needle device can comprise blunt ends, in which case stylets having pointed ends are preferably inserted into the needle device in order to facilitate the insertion of the needle device into the vasculature of a subject. Such needle devices can also preferably comprise one or more openings on the side wall of the needles. In a further embodiment, the present needle device can be used to perform biopsies.

### DRAWINGS

These and other features, aspects and advantages of the present invention will become better understood with regard to the following description, appended claims, and accompanying figures where:

FIG. 1 is a perspective view of an embodiment of the present lancet.

FIG. 2 is a top plan view of the needle retaining surface of the lancet of FIG. 1.

FIG. 3 is a side view of the lancet of FIG. 1.

FIG. 4A is a perspective view of a lancet having a protective top.

FIG. 4B is an exploded perspective view of the lancet of FIG. 4A.

FIG. 5 is a perspective view of a lancing device for use with a lancet of the present invention.

FIG. 6 is a perspective view of a multiple-needle biopsy device attached to a syringe.

FIG. 7 is a rear perspective view of the biopsy device illustrated in FIG. 6.

FIG. 8 is a perspective view of stylets for use with the biopsy device of FIG. 7.

FIG. 9 is a rear perspective view of the stylets of FIG. 8 inserted into the biopsy device of FIG. 7.

FIG. 10 is a perspective view of a syringe with 2 connecting tips.

FIG. 11 is a perspective view of a syringe with 2 connecting tips wherein each connecting tip is mated with a needle device having a single needle mounted on a single needle hub.

FIG. 12 is a perspective view of a syringe with a single tip that is mated with a needle device having 2 needles mounted on a single common needle hub.

FIG. 13 is a perspective view of a needle device wherein a single needle is mounted on a single needle hub.

FIG. 14 is a perspective view of a needle device wherein 2 needles are mounted on a single common needle hub.

FIG. 15 is a side plan view of a syringe device having 2 small chamber lumens on a common larger barrel, wherein each small chamber lumen has 2 connecting tips.

FIG. 16 is a rear perspective view of the syringe device of FIG. 15.

FIG. 17 is perspective view of a hemodialysis needle device having 2 needles mounted on a needle hub.

FIG. 18 is a partial cutaway view of a forearm of a patient undergoing hemodialysis using the needle device of FIG. 17.

FIG. 19 is a perspective view of a different embodiment of a hemodialysis needle device coupled with a stylet device.

FIG. 20 is a perspective view of the needle device of FIG. 19 after removing the stylet device.

FIG. 21 is a perspective view of a stylet device for use with the needle device of FIG. 20.

FIG. 22 is a perspective view of a hemodialysis needle device without a wing for gripping.

FIG. 23 is an exploded, perspective view of the needle device of FIG. 22 having a spiral lock in the posterior opening to secure a connection with a connecting catheter having mating spiral grooves.

All dimensions specified in this disclosure are by way of example only and are not intended to be limiting. Further, the proportions shown in these Figures are not necessarily to scale. As will be understood by those with skill in the art with reference to this disclosure, the actual dimensions of any device or part of a device disclosed in this disclosure will be determined by their intended use.

### DESCRIPTION

### Definitions

As used herein, the following terms and variations thereof have the meanings given below, unless a different meaning is clearly intended by the context in which such term is used.

“Lancet” means a device comprising one or more points or blades that are capable of puncturing a skin surface of an individual in order to obtain a sample of blood.

“Lancing device” means a device which retains a lancet and which is capable of projecting the lancet through a skin surface of an individual.

“Lumen,” with respect to a part of a human or animal body, means a cavity or channel within an organ, in particular a tubular organ such as a blood vessel or the intestine.

“Stylet” means a hollow needle adapted to be telescopically interposed within another needle or cannula.

As used herein, the term “comprise” and variations of the term, such as “comprising” and “comprises,” are not intended to exclude other additives, components, integers or steps. The terms “a,” “an,” and “the” and similar referents used herein are to be construed to cover both the singular and the plural unless their usage in context indicates otherwise.

### Lancets

The present lancets comprise multiple sharp projections, preferably needles, for penetrating the skin of an individual and drawing blood from the wound inflicted by such needles. The needles used in the present lancet are preferably half the diameter of the needles used in comparable single-needle lancets or less. In an application in which a single, thicker needle would be used by an individual to draw a desired amount of blood, a lancet as provided herein having at least two parallel, spaced-apart needles, each preferably having half the desired diameter as compared to the single thicker needle, can be used. The amount of blood produced by the single larger (thicker) needle is going to be similar to or approximately the same as the amount of blood produced by the use of two needles, each of which is half the diameter of the larger needle. However, the amount of pain experienced by the individual when using a multiple needle lancet will be less.

In addition to reducing the pain suffered by individuals who need to obtain a blood sample with a lancet, the use of the present multiple needle lancets results in faster healing times for such individuals as compared to the use of a single needle used to draw an equivalent amount of blood. This is because such larger wounds heal more slowly than smaller wounds, and the tissue healing time of a puncture wound is dependent on the size of the puncture and not on the number of punctures inflicted. For example, a puncture wound on the skin by a 14 gauge needle requires more time to heal than 3 different puncture wounds by a 21 gauge needles. This is because the body's healing process takes place separately for each wound. If it takes 7 days to heal the wound by the bigger needle, it would typically take 4 days to heal all 3 wounds made by the smaller needles. Thus, the present inventor has found that multiple small needle wounds in proximity to one another heal more quickly than a single large wound inflicted by a larger needle.

Preferred lancets according to the present invention have needles made of metal, ceramic, or plastic, at least one end of which is pointed (i.e., the tip portion 9). Commercially available needles of various diameters can be used in the present lancet, preferably needles of at least 34 gauge, more preferably needles of between 36 gauge and 44 gauge, and more preferably 40 gauge needles are used. Needles having smaller diameters inflict less pain and smaller wounds which heal more quickly as compared to larger diameter needles. Larger numbers of smaller diameter (thinner) needles for obtaining a blood sample of a desired volume are preferred over smaller numbers of larger diameter needles.

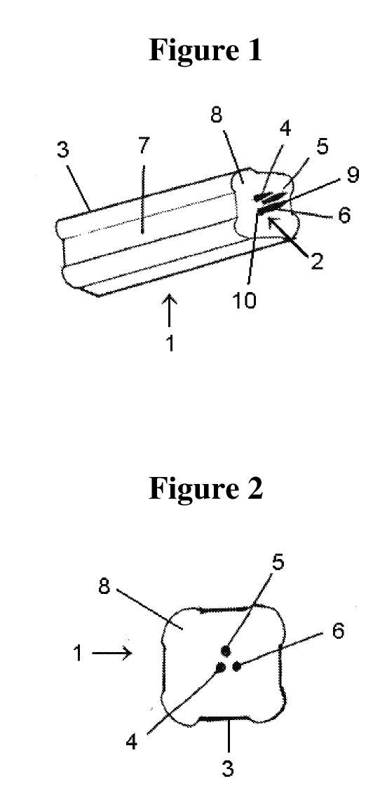

The needles 2 can be either solid or hollow, though solid needles are preferred for lancing applications in which fluids are not withdrawn through such needles. Needles 2 used in the present lancet 1 are attached to and project outwardly from a lancing body 3. Each of the needles 2 is arranged in a parallel configuration with respect to each of the other needles 2 retained by the lancing body 3. Although each needle 2 is approximately parallel to each other individual needle, in embodiments in which three or more needles 2 are used in a lancet 1, the needles are preferably arranged in a nonlinear fashion in order to provide a more compact arrangement of the needles 2 on the needle retaining surface 8 of the lancet body 3, as illustrated in FIGS. 1 and 2. The needles 2 preferably project from the lancet body 3, and in particular from a needle retaining surface 8 of the lancet body 3, by distances of between about 1 mm. (millimeters) and 3 mm.

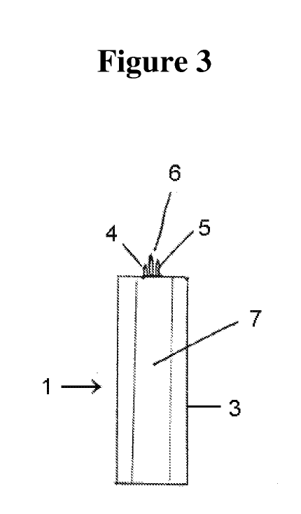

In one embodiment, the needles 2 of the lancet 1 project from the lancet 1 by different distances, so that the tips 9 of the needles 2 are adapted to penetrate the skin of a patient sequentially when the lancet 1 is urged toward a skin surface which is approximately perpendicular to the direction of the motion the lancet 1. In embodiments in which the needle retaining surface 8 of the lancet body 3 is approximately perpendicular to the longitudinal dimension of the needles 2, as shown in FIGS. 1-3, the needles have different lengths and thereby project from the needle retaining surface 8 of the lancet body 3 by different distances. Alternatively, the needle retaining surface 8 can comprise a non-planar shape or can contact a base portion 10 of the needles at a non-perpendicular angle. In such an embodiment, the needles (which are still arranged parallel to one another) are preferably arranged parallel to a guide groove 7 or other structure of a lancet 1 or a lancing device 20 that retains the lancet 1 which serves to guide the lancet toward a skin surface when the lancet 1 is used to obtain a blood sample. Such a groove 7 or other structure preferably has a shape or configuration which is parallel to the direction of motion of the lancet 1 when the lancet 1 is urged toward a skin surface. In this embodiment, the needles 2 of the lancet 1 project from the lancet 1 by different distances with respect to a predetermined point along the longitudinal axis of the lancet body 3, i.e. the axis parallel to the needles 2.

A lancet according to this embodiment is shown in FIGS. 1-3. As illustrated, the needle 4 projects a shorter distance from needle retaining surface 8 than needle 5, which itself is shorter than needle 6. In one example, such a lancet 1 can comprise three 36 gauge needles which project by 1 mm., 1.5 mm., and 2 mm., respectively, from the needle retaining surface 8 of the lancet body 3.

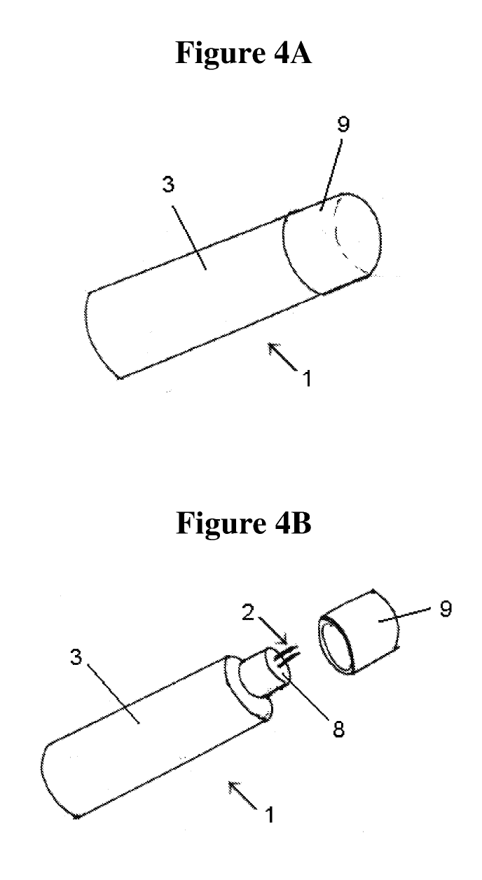

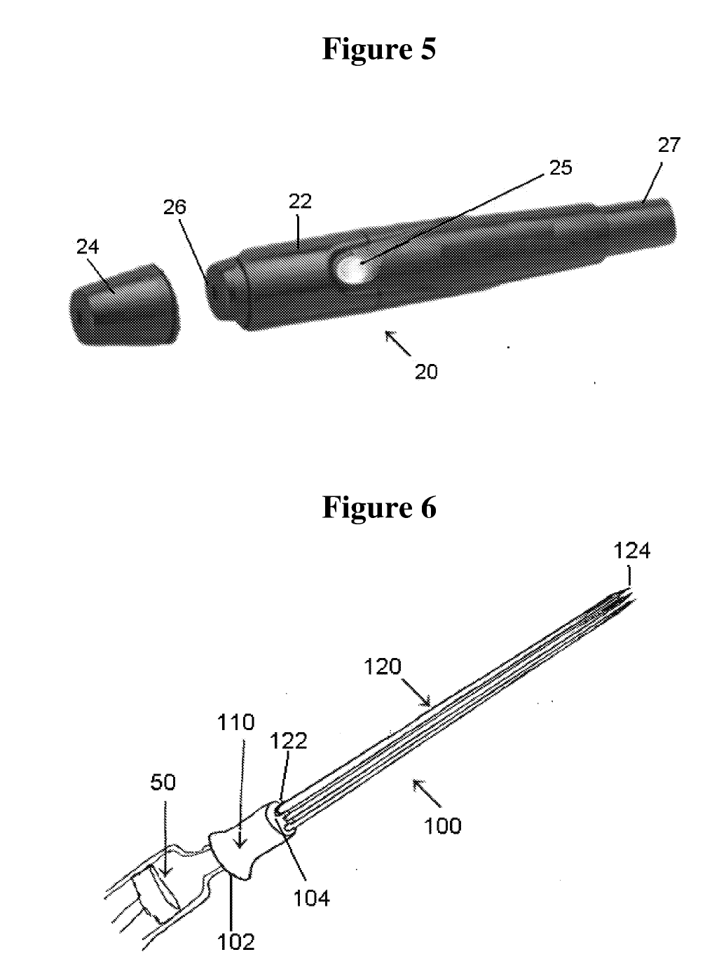

In another embodiment, the needles 2 of the lancet 1 project from the lancet 1 by approximately the same distance, so that they can puncture a skin surface at approximately the same time. This embodiment is illustrated in FIG. 4B, in which the needles 2 project by approximately the same distance from a needle retaining surface 8 of the lancet body 3, which is approximately perpendicular to the longitudinal dimension of the needles 2. In this embodiment, the tips 9 of the needles 2 lie in approximately the same plane, and this plane is preferably approximately perpendicular to the direction of motion of the lancet 1 when the lancet 1 is urged toward a skin surface.

In a preferred embodiment, the rear part of a lancet needle 2 facing away from this tip is wholly or partially enclosed in a lancet body 3, which is preferably made of plastic. Such a lancet body 3 can be manufactured by positioning the lancet needles 2 in a plastic injection mold and injection molding the lancet body 3. In this process, a protective sheath 9 made of plastic can also be injection molded at the same time over the tip of the lancet, if desired. A protective sheath 9 can alternatively be formed separately from the lancet body 3 and then attached, as shown in FIGS. 4A and 4B.

The lancet body 3 is preferably provided with a shape which allows it to be gripped and retained by a lancing device 20. In the embodiment shown in FIGS. 1-3, the lancet body 3 has a pair of guide grooves 7 which can be engaged by complementary shaped projections of a lancet holder 26 of the lancing device 20 in order to grip the lancet. Alternatively, projections on the lancet body 3 can engage corresponding recesses in the lancet holder 26 of the lancing device 20. The lancet body 3 preferably conforms to a commercially standard size, e.g. 22 mm in length and 6 mm in diameter, although other sizes are possible so that the present lancet can be adapted to fit the different lancing devices of various manufacturers.

In an alternative embodiment, the lancet holder 26 can be adapted to retain a plurality of lancets 1. Individual lancets 1 in this embodiment can be linked together to form a set of lancets 1, for example in the region of their tips or lancet bodies 3.

### Lancing Devices

A variety of commercial lancing devices can be used in the present needle system, such as the SOFTCLIX II device (available from Boehringer Mannheim GmbH, Mannheim, Germany), the BD Lancet Device (available from Becton, Dickinson and Company, Franklin Lakes, N.J., USA), or the PENLET PLUS device (available from LifeScan, Milpitas, Calif., USA). In one embodiment, the lancing device 20 can comprise an elongate cylindrical housing 22 having essentially the shape of a pen. Lancing devices 20 include in their interior an ejector, i.e. a mechanism which guides a lancet 1 in the lancing process towards and, after the lancing, away from the desired lancing site on the skin of a user of the device. This mechanism can be driven, e.g., by a manually tensioned spring. A triggering button 25 can be present on the housing 22 of the lancing device 20 in order to trigger the mechanism. Lancing devices 20 further preferably comprise a cap 24.

Such pen-shaped lancing devices preferably include a depth adjustment control knob 27. When a deeper puncture is desired, to produce a greater amount of blood, the knob 27 can be adjusted in order to make the needle penetrate the skin deeper. In one embodiment, the length of one or more needles mounted on a lancet can be different from one or more other needles. This is because some patients have more delicate and/or thinner skin that requires only a single puncture with a thin needle to obtain enough blood for testing. On the other hand, other patients have thicker skin which may require 2 or more simultaneous needle punctures for adequate blood sampling using the thinner needles of the present lancets. By adjusting the depth adjustment control knob 27 of the lancing device 20, patients can have a choice of one needle puncture or 2 or more simultaneous punctures.

Although the present lancets 1 are typically made as single-use items, in order to prevent infections and the spread of disease, the lancing devices 20 can be reusable, in which case the lancing devices 20 reversibly retain the lancets 1, which can be removed after use and replaced with an unused, clean lancet 1. In some embodiments, a plurality of lancets 1 can be stored in a lancing device 20 and can be automatically placed into position for use in the lancing device 20 upon removal of a used lancet 1. Alternatively, such used lancets can be retained in a portion of the lancing device 20 for later removal by a user. In an alternative embodiment, the lancing device 20 and lancet 1 can be combined as a single disposable device, whereby the lancet 1 and ejector mechanism are manufactured together as a single device, in which case the lancet 1 is not removably secured to the lancing device 20.

### Method of Using Lancets

A lancet 1 with multiple thin needles 2 can be used to puncture the skin of a patient either manually by hand, or mechanically by a lancing device 20. A patient first cleans the skin of the area of skin which is to be punctured by the lancet 1. When used manually, the lancing body 3 is held with the fingers and the needles 2 are poked into the skin. When used mechanically, the lancet body 3 of the lancet 1 is placed into a lancet holder 24 of a lancing device 20. By actuating the trigger 25 of the lancing device 20, the needles 2 hit and puncture the skin of a user of the device 20.

When using a lancet 1 according to the embodiment illustrated in FIGS. 1-3, the lancet 1 and lancing device 20 can be adapted to withdraw different amounts of blood depending on the number of needles 2 which puncture the skin of a subject and the depth to which such needles 1 penetrate. When such a lancet 1 is used with a lancing device 20, the depth adjustment control 27 is set to project the lancet 1 by a predetermined distance which correlates to a predetermined puncture depth by the needle or needles 2. If only an amount of blood drawn by a single needle is desired, then the depth adjustment control 27 is set to project the lancet 1 by a distance such that only the longest needle 6 punctures the skin. If a larger blood sample is needed, the control 27 can be adjusted to provide for both needles 5 and 6 to puncture the wound (thereby also causing the longest needle 6 to puncture the skin more deeply and likely withdraw more blood). Greater amounts of blood can likewise be drawn by further adjusting the depth adjustment control 27 and/or by otherwise urging the lancet 1 to puncture the skin of a subject by a greater distance and/or with a greater number of needles 2.

After lancing, a small droplet of blood may appear spontaneously at the lancing site, usually 2-20 μl in volume. Otherwise, blood samples can then be obtained by gently squeezing the pricked skin. The area of the skin punctured by a lancet 1 can be the finger, palm, heel, foot, earlobe, or any part of the body where a desired blood sample can be obtained. Once an adequate amount of blood for a particular test has been obtained, the sample can be subjected to testing, such as on a test strip for use with a glucose meter. Other analytical tests for determining other constituents or properties of a blood sample can also be performed with a blood sample obtained with the present lancets.

Biopsy Device with Multiple Needles

In another aspect, the present invention can comprise a biopsy device comprising multiple needles. Biopsies are conducted when a patient is suspected of having a certain disease, such as cancer, in which case a sample of tissue from a suspicious lesion is obtained with a biopsy needle. There are two types of biopsy needles, aspiration biopsy needles and core biopsy needles. Aspiration biopsy needles are usually thin and are used for aspirating tissue or cells, while core biopsy needles are thicker and are used for obtaining a larger amount of tissue.

The present biopsy needle device can be used for injecting or aspirating fluid. In one example, the present biopsy device can have 2 very thin needles instead of 1 thicker needle to aspirate a body fluid. Alternatively, the present biopsy device can comprise a plurality of core biopsy needles for obtaining solid or semi-solid tissue samples from a subject. The present biopsy device with multiple thinner needles causes less tissue injury due to the use of small sized needles, while at the same time it can obtain more tissue samples over a broader area because of the use of a plurality of needles, thereby providing a better chance of acquiring more accurate tissue samples.

The present biopsy device 100 comprises a plurality of thin hollow needles 120, each having a proximal end 122 and a distal end 124, which are attached to a means for connecting the thin hollow needles 120 to an instrument, such as a stylet 150 or syringe 50, in a secure manner. The means for connecting can comprise a connector 110 having a body portion 105 comprising a proximal end 102 and a distal end 104. The needles 120 are arranged parallel to each other, and can be arranged on the distal end 104 of the connector 110 in either a non-linear fashion (as illustrated in FIG. 6) or in a linear manner.

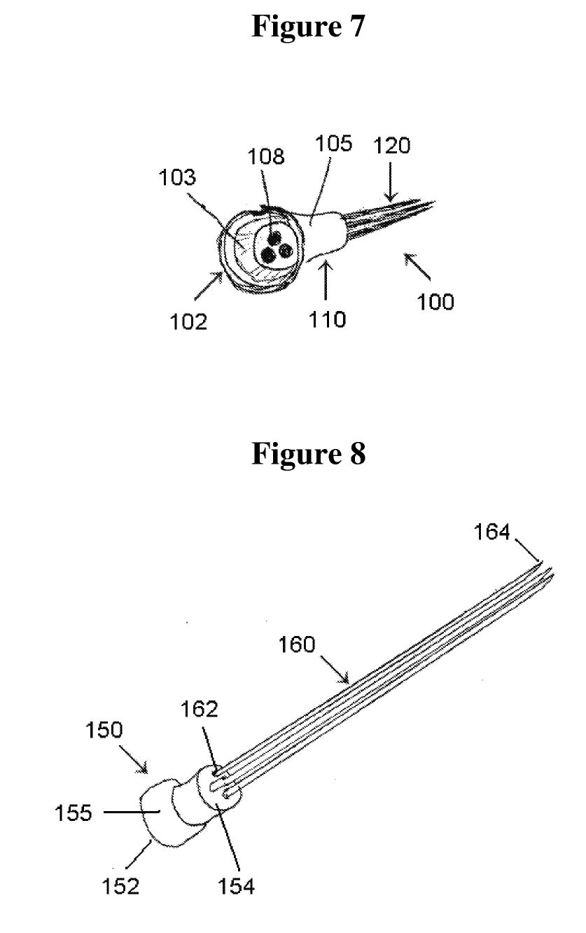

FIGS. 6 and 7 are perspective views of a biopsy device 100 in which a syringe 50 can be attached to the connector 110 in order to withdraw biopsy tissue samples from the hollow biopsy needles 120. In the interior 103 of the connector body 105 are openings 108 that are connected directly with the tunnels of the hollow biopsy needles 120. A syringe 50 or stylet device 150 can be fitted into the interior area 103 of the biopsy device 100 and secured to the connector body 105.

The stylet 150 comprises a connector body 155 having a proximal end 152 and a distal end 154 and a plurality of stylet needles 160. The distal ends 164 of the stylet needles 160 are inserted into the hollows of the biopsy needles 120 through the openings 108 and the proximal ends 162 of the stylets are urged toward the openings 108 before puncturing the skin of a subject. A stylet needle 160 occupies the tunnel of each biopsy needle 120. Once the insertion is complete, the member of stylets is secured by locking in a notch 101. Once the combined needles 120 and 160 reach the target lesion, then the stylets 120 are removed to make the biopsy needle hollow in order to obtain the tissue samples.

In one embodiment, the tips or distal ends 124 of the needles 120 project from the distal end 104 of the connector 110 by a different distance. By making the length of the needles slightly different, the area of biopsy can also be expanded, i.e. can be diversified in three dimensions, thus further reducing the probability of false negative results.

Method of Using a Biopsy Device with Multiple Needles

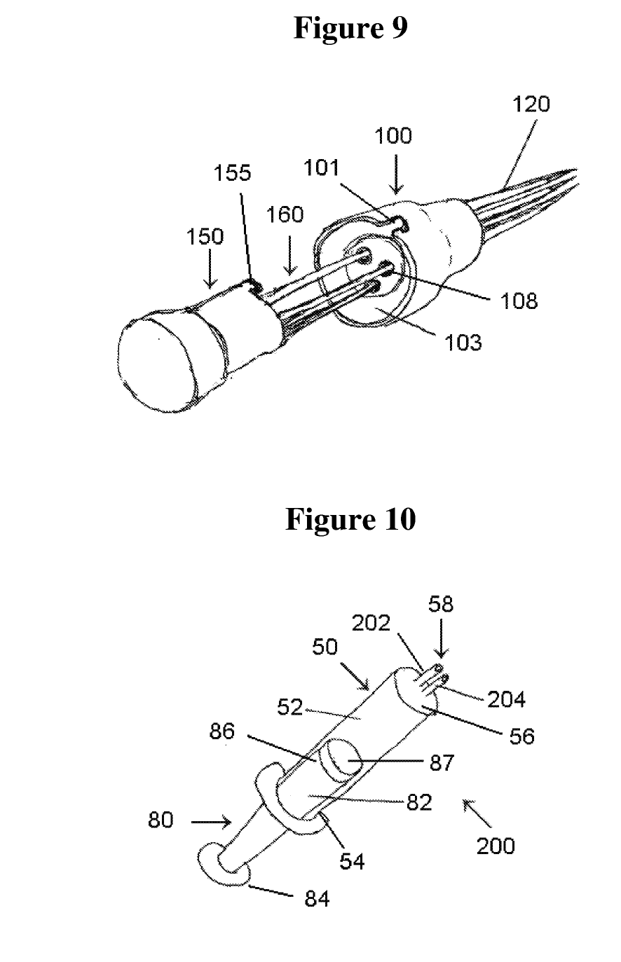

Before the needles 120 of the present biopsy device 100 are introduced into a subject, such as a person or an animal, the biopsy device 100 and the stylet device 150 are joined together by inserting each stylet 160 into a corresponding hollow of a biopsy needle 120 through the posterior opening holes 108 (as illustrated in FIG. 9). This joining of the needles 120 and stylets 160 is secured by securing a locking member 155 of the stylet device 150 into the notch 101 located in the rim of the connector 110 of the biopsy device 100. A user then punctures the skin of a desirable area using the joined needles and keeps inserting them until the tips 124 of the needles reach the target lesion. Usually, this procedure is done with help of a CT scan. The user can confirm that the location of the needles is appropriate by watching the CT scan screen. Once confirmed, the stylet needles 160 are removed from the biopsy needles 120 to make the hollow biopsy needles patent. Then the user pushes and pulls back the needles 120 several times slightly while the needles are placed inside the target lesion. Because the needles have patent hollows, the tissue samples are filled inside the hollows by this method. An empty syringe 50 can then be tightly placed into the connector 110 of the biopsy needle device 100 in order to withdraw the tissue samples into the syringe 50. A pathologist can then remove the tissue samples for examination

Syringe Device with Multiple Needles

In another aspect, the present invention comprises a syringe device having multiple thin needles instead of a single large needle. Such a system is less painful when puncturing the skin and other organs, causes less tissue injury, and results in faster healing time, while still allowing a sufficient amount of liquid to be delivered (or withdrawn) efficiently through the bores of the multiple needles. The diameter or gauge of the needles can be any size typically used for injecting or withdrawing fluids from a subject, normally between 18 and 31 gauge, although thinner needles such as 40 or 45 gauge needles could also be used, with the appropriate needle size for a particular application or treatment using the present syringe device depending on the amount of fluid to be delivered or withdrawn and on the number of needles used.

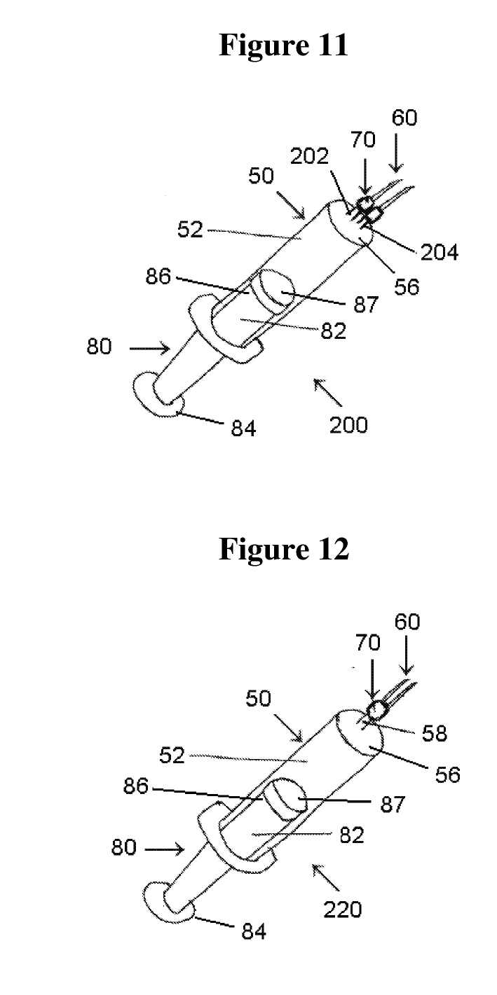

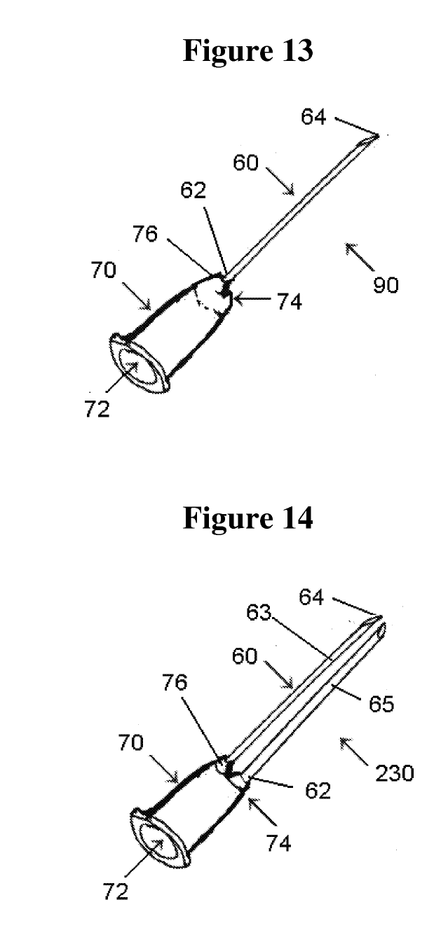

The syringe portion 50 of the present syringe device generally comprises a barrel 52 having a proximal end 54 for engaging a plunger 80 or other means for creating either positive or negative pressure in the barrel 52, and a distal end 56 comprising a connecting tip 58 for attachment to the proximal end 72 of a needle hub 70. The plunger 80 comprises a shaft 82 having proximal end 84 for actuation by a user of the syringe and a distal end 86, which may comprise a stopper 87 or other means for retaining a liquid within the barrel 52 of the syringe 50. In one embodiment, shown in FIGS. 10 and 11, the distal end 56 of the barrel 52 comprises two connecting tips 58 (tips 202 and 204) for engaging two needle hubs 70. As shown in FIGS. 11 and 13, the needle hubs 70 can each comprise a single needle seat 76 for retaining the proximal end 62 of a needle or cannulae 60, which comprises a sharp point at its distal end 64.

In an alternative embodiment, shown in FIGS. 12 and 14, the needle hub or hubs 70 can include two needle seats 76 for retaining two needles 60. Each of the needles (63 and 65) is disposed parallel to each other, and also preferably parallel to the hub 70 and to the barrel 52, when the hub 70 is connected to the connecting tip 58 of the barrel 52. In this way, a standard syringe with a single connecting tip 58 can be adapted to dispense or draw fluids through two needles 60. The dual needle hub 70 of FIG. 14 can also be used in connection with the dual connecting tip syringe 50 of FIG. 11, thereby enabling fluids to be dispensed or withdrawn from four needles 60, in which case the needles will preferably be disposed so as to be both parallel to each other and arranged in a linear fashion. One of skill in the art will appreciate that different numbers of connecting tips 58, hubs 70, and needles 60 can be configured for particular purposes.

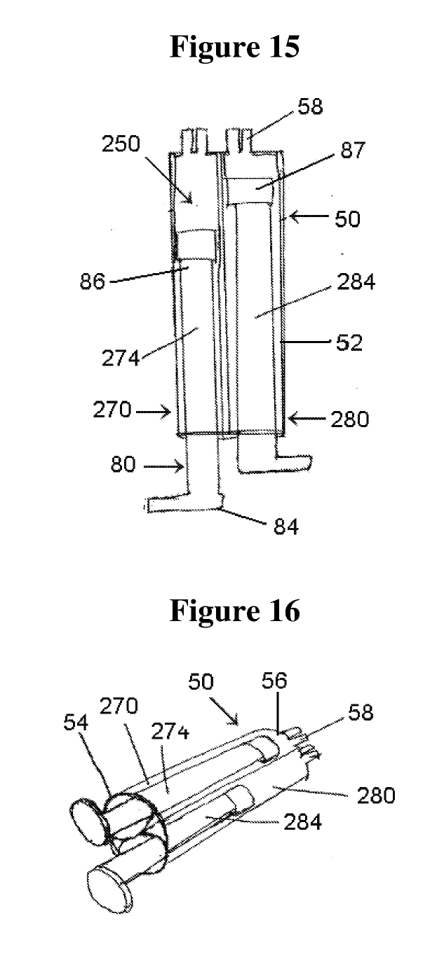

In a further embodiment, the barrel 52 of the syringe portion 50 can be comprised of a plurality of smaller chambers 250 within the barrel 52, and each chamber 250 can comprise a separate plunger 80 or other means for transferring a fluid material in or out of the chamber 250. In the embodiment shown in FIGS. 15 and 16, the syringe portion 50 comprises two chambers 270 and 280, each of which comprises two connecting tips 58. In this embodiment, a larger number of needles can be attached to the syringe device. In addition, a plurality of different liquid materials can be injected simultaneously or sequentially through the multiple chambers and needles.

In the foregoing embodiments, the connecting tip 58 of the barrel 52 is preferably adapted to mate securely with the proximal end 72 of the needle hub 70. In a preferred embodiment, the connecting tip 58 and needle hub 70 are reversibly secured to one another via male-female interfaces, threaded interfaces, or other connection means known to the art. Alternatively, the connecting tip 58 and needle hub 70 can be permanently secured, such as with adhesive, or can be molded or otherwise constructed as a single piece.

Method of Using a Syringe Device with Multiple Needles

The present syringe devices can be used to administer fluid materials, such as vaccines or other therapeutic agents, to a subject, in particular a human subject. The syringe devices can be used or adapted to be used to administer such materials intravenously, intraperitoneally, intramuscularly, intravitreally, intrathecally, or in other manners known to the art for delivering a liquid to a lumen or non-lumen space of a subject.

In use, a needle device, such as either needle device 90 of FIG. 13 or the needle device 230 of FIG. 14, is attached to one or more connecting tips 58 in order to provide a plurality of needles on a syringe device 50. The syringe barrel 52 is filled with a liquid material by transferring the liquid from a container, such as a drug vial, by pulling the plunger 80 backward, i.e. such that the distal end 86 of the plunger 80 moves away from the distal end 56 of the barrel 52. A different large bore needle can be used for the purpose of transferring the liquids from the container. After filling the barrel 52 with a desirable amount of the liquid, the needle 60 will puncture the target object, such as the skin of a subject. Once the needle 60 penetrates the target object to a desired depth, the plunger 80 is pushed forward to inject the liquid out of the barrel 52, transferring the liquid to the target object. Once the desired amount of the liquid is injected into the target object, the whole system with the syringe and the needles is withdrawn from the target object.

The present syringe device can also be used in the opposite order of steps in order to withdraw a fluid from a patient. That is, the plunger 80 can first be actuated, after which the needles 60 of the syringe device can be inserted into a target location of a subject, and fluid then withdrawn by pulling the plunger in the opposite direction.

In a syringe system such as that of FIGS. 15 and 16, each small chamber lumen 250 can be filled with a different type, concentration, or formulation of liquid material. After each chamber 250 is filled with the liquids, a single puncturing action with the syringe having multiple needles is made. Once the needles 60 have reached the target object, such as a vein or artery, the plunger of each chamber barrel is pushed forward to transfer the liquids out of the chambers 250, either simultaneously or sequentially, depending on the therapeutic modality indicated or desired when using such liquid materials.

Hemodialysis Device with Multiple Needles

When kidney function fails due to various reasons, such as diabetes mellitus or atherosclerosis, a subject's ability to excrete bodily wastes through the urine becomes diminished or completely impaired. One of the most common treatment procedures for getting rid of such wastes is hemodialysis. The principle of hemodialysis is to artificially filter the blood of the patient containing the bodily wastes with a hemodialysis machine. Through a large needle, the patient's blood is drained out of the arteriovenous access that is usually created in the arm surgically, i.e. an arteriovenous fistula (420, FIG. 18) or an arteriovenous graft. After the blood has been filtered by the hemodialysis machine, it will be infused back to the patient through another large needle that is placed into the arteriovenous access at a different site from the needle for blood draining.

The needles currently used for arteriovenous access for hemodialysis has a size of 14 to 15 gauge, and patients with kidney failure usually undergo hemodialysis 3 times a week. Because a large-sized needle is used to puncture the delicate wall of the arteriovenous access and because hemodialysis is administered frequently, patients suffer not only pain but also other problems from tissue injuries and damage to the arteriovenous access, such as thrombosis, infection, and the malfunction and/or closure of the arteriovenous access. The cost of treating such problems as well as the pain and suffering of patients is tremendous.

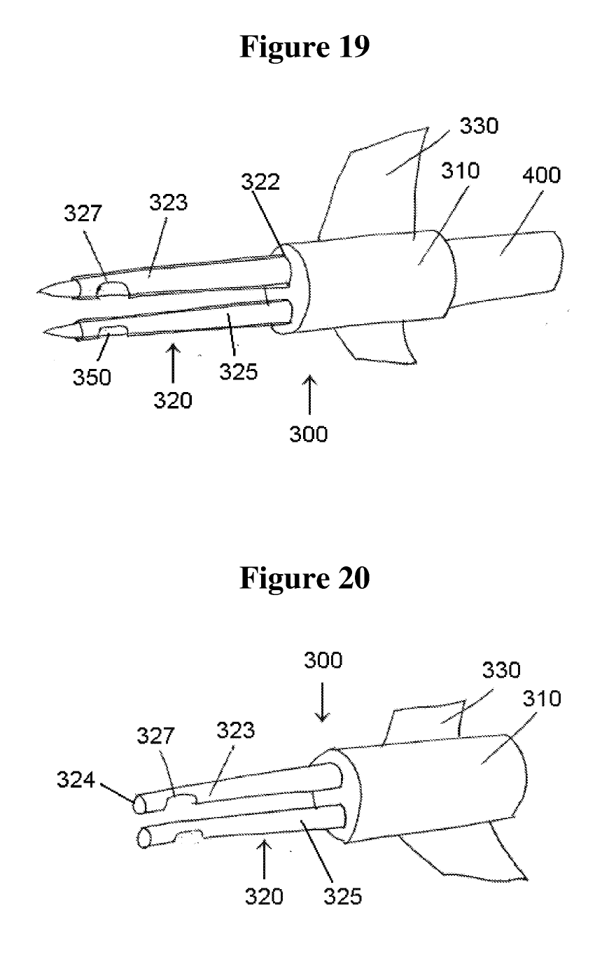

The present hemodialysis needle system can comprise needles 320 of various sizes, lengths, shapes, and materials. For example, the needle 320 can be a cannula made of a plastic material. Such needles 320 can have various shapes, with or without cutting edges. For example, an extra opening 327 can be created on the side wall of the needle 320 to reduce jet pressure and to increase the blood flow rate (FIGS. 19 and 20). A stylet device 350 can also be coupled with the needle device 300 as shown in FIG. 19. Accessories such as an attached wing 330 can also be included on the needle device 300 for gripping and controlling it. The present hemodialysis needle system can also be used to puncture other parts of the body to drain or inject liquids, such as for venous puncturing, arterial puncturing, or puncturing other organs such as lung, liver, peritoneum, pleural membrane, or the eyeball.

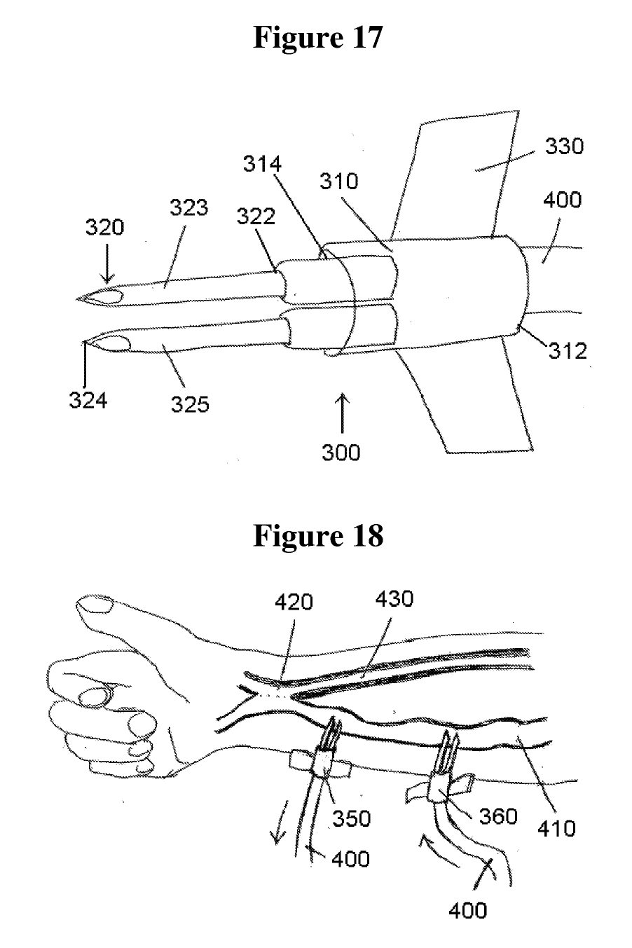

FIG. 17 illustrates a hemodialysis needle device 300 having two needles 320 mounted on a needle hub 310. The needles are preferably 18 gauge, though other sized needles can also be used, preferably needles of between approximately 16 and 20 gauge. Through the use of 2 needles that are 18 gauge, for example, the overall blood flow rate would be comparable to or even better than that of a single 15 gauge needle. The needles 320 on the hub 310 are preferably spaced between about 5 mm. and 15 mm. apart from each other. While the illustrated embodiments involve the use of two needles mounted to a hub (i.e., the needles 323 and 325 in FIGS. 17, 19, 20 and 22), in alternative embodiments additional needles could be used, preferably arranged in a linear fashion on the hub 310.

The needles 320 each comprise a proximal end 322 attached to distal end 314 of the needle hub 310, as well as a distal end 324 for insertion into the vasculature of a subject. The distal end 324 can be sharp, i.e. have a cutting edge as shown in FIG. 17, or alternatively can be blunt, as shown in FIG. 19. A plastic wing 330 can be attached to the needle hub 310 for gripping and controlling the needle device 300.

The placement and use of the present hemodialysis needle device 300 is illustrated in FIG. 18, which depicts the forearm of a patient undergoing hemodialysis. An arteriovenous access 420 joining an arterial blood flow 430 with a venous blood flow 410 is preferably provided upstream of where the hemodialysis needle device 350 is inserted for draining the blood at a very high blood flow rate. Another needle device 360 is provided with access to the venous side of the arteriovenous access 420, i.e. downstream of the needle device 350, to infuse back the dialyzed (filtered) blood to the patient. The flow of blood through the conduits 400 connecting the needle devices 350 and 360 to a dialysis machine is illustrated with arrows in this figure. In order to dialyze the blood within several hours, the blood flow rate through the needles has to be high.

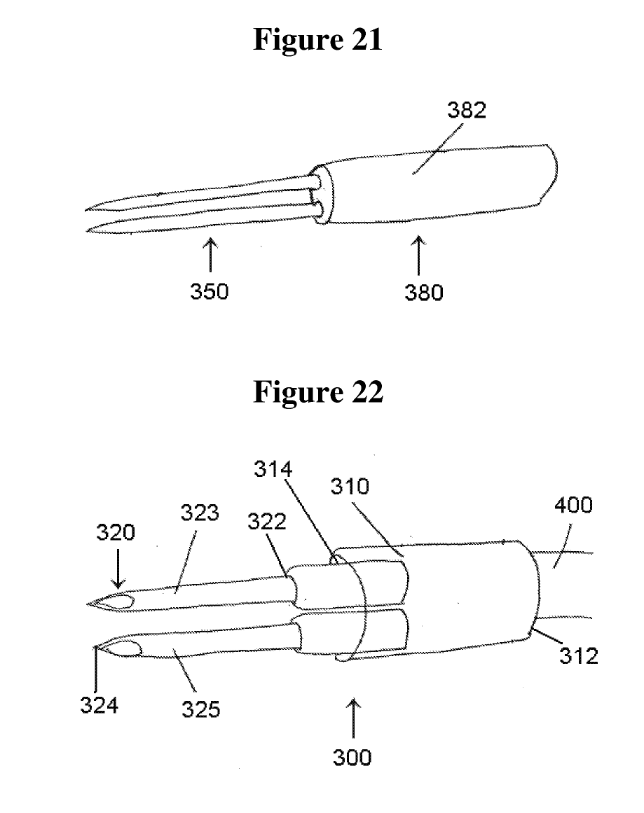

FIG. 19 illustrates a different embodiment of the present needle device 300. In this embodiment, a stylet device 350 is used to couple the needle device 300 to minimize the tissue injury by the cutting edge at proximal end 324 of the needle 320 when puncturing the skin and vasculature of a subject. The tip 324 of the needle 320 in this embodiment is rounded and does not comprise a cutting edge. This needle device 300 also has an opening 327 in the side wall of the needle to reduce the jet pressure from the high blood flow rate by reducing resistance of the lumen, and to increase the blood flow rate. A plurality of such openings can also be provided in the needle 320 to further improve blood flow. FIG. 20 shows the needle device of FIG. 19 after the stylet device has been removed, while FIG. 21 illustrates a stylet device 380 for use with the needle device of FIG. 20. The depicted stylet device 380 has 2 stylet needles 350 mounted on the body part 382.

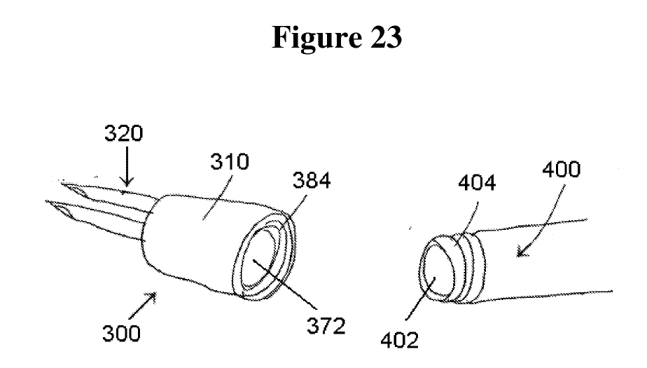

FIG. 22 illustrates an alternative embodiment of the present needle device which lacks a grip wing 330. This needle concept can be used for other purposes, such as for venous puncture, arterial puncture, or for injecting liquids into other target organs. FIG. 23 further illustrations the connecting of a fluid conduit 400 that can be attached to the needle device 300, such as through the use of a spiral lock 384 in the posterior opening 372 of the needle hub 310 that can be secured to mating threads 404 in a distal end 402 of the conduit 400.

Method of Using a Hemodialysis Device with Multiple Needles

In order to perform hemodialysis with the present hemodialysis needle device 300, the device 300 is first attached to a conduit 400 leading to the intake of a dialysis machine, and the skin where the device 300 is to be inserted is prepared by cleaning it. A practitioner such as a nurse then preferably grips the wings 330 of the needle device 300 and inserts it into the venous side of the arteriovenous access 420 (e.g., as shown by needle device 350 in FIG. 18) as the draining needle. Another needle device 300 is inserted to infuse the blood back after hemodialysis (e.g., needle device 360 in FIG. 18). The blood drained out of the patient through the draining needle and the connecting catheter is sent to the dialysis machine for filtering the bodily wastes. The dialyzed blood is later then infused back to the patient through the infusing needle. The needles are withdrawn when the hemodialysis procedure is completed.

Using the embodiment of the needle device of FIG. 19, the stylet (shown in FIG. 21) is pulled back and removed from the needle device 300 after the needle device 300 is inserted into a vein. By avoiding the use of a cutting edge on the needle 320, tissue injury can be minimized. A side wall opening (327) is created to reduce the blood flow resistance and jet pressure.

### EXAMPLES

### Example 1

### Use of a Multiple Needle Lancet Device

A lancet device having 2 needles mounted on a plastic lancet body is provided. The needles have lengths of 1.5 mm and 2 mm as measured from the surface of the lancet body from which they project, respectively. The lancet is placed in a lancing device, and the depth adjustment control knob of the lancing device is adjusted so that both needles will puncture the skin of the individual using the lancing device. The individual places the lancing device on a portion of his skin and actuates the release button of the lancing device, thereby urging the needles forward. Both needles puncture the skin of the patient and produce blood.

The patient removes the used lancet from the lancing device and replaces it with an unused lancet. The depth adjustment control knob of the lancing device is now adjusted so that only the longer needles will puncture the skin of the individual using the lancing device. The individual places the lancing device on a different portion of his skin and actuates the release button of the lancing device, thereby urging the needles forward. Only the longer needle punctures the skin of the patient and produces blood.

### Example 2

### Use of a Multiple Needle Biopsy Device

A 1 cm tumor lesion is found in the right lung of a patient in a CT (Computer Tomography) scan. It is necessary to obtain the tissue sample by a needle biopsy to find out whether it is cancerous or not. A radiologist employs a biopsy device as shown in FIGS. 6-9 and described above having 21 gauge biopsy needles and introduces the needles manually into the lung through the skin of the chest while a watching the CT scan. The CT scan shows the needles in the area of the suspected lung tumor. Once it is confirmed that the biopsy needle has hit the tumor precisely, the radiologist passes the needles through the lesion in order to obtain tissue samples of the tumor. The tissue samples are caught in the biopsy needle and are then sucked out by a syringe attached to the other end of the biopsy needle device.

### Example 3

### Use of a Multiple Needle Syringe for Intravitreous Injection

A topical anesthesia is first used to numb the outer layer of the eyeball of a subject. An ophthalmologist fills a syringe as shown in FIG. 11 or 12 with a therapeutic agent for the treatment of macular degeneration. The ophthalmologist attaches two needles each with a size of 33 gauge to the syringe and then injects the therapeutic agent into the eyeball of the subject (intravitreous injection).

### Example 4

### Use of a Multiple Needle Syringe for Intravenous Injection

A physician fills a syringe as shown in FIG. 11 or 12 with a therapeutic agent for the treatment of a medical condition. The physician attaches two needles each with a size of 33 gauge to the syringe and then inserts the needles into a vein of the subject. The physician then injects the therapeutic agent into the vein of the subject.

### Example 5

### Use of a Multiple Needle Syringe for Intramuscular Injection

A physician fills a syringe as shown in FIG. 11 or 12 with a vaccine. The physician attaches two needles each with a size of 33 gauge to the syringe and then inserts the needles into the musculature of a subject. The physician then injects the vaccine into the subject.

### Example 6

### Use of a Multiple Tip Syringe Device

In a medical procedure called paracentesis, a syringe mounted with a hypodermic hollow needle is inserted into the abdominal cavity where ascites fluid is collected by puncturing the abdominal wall skin. A 23 gauge hypodermic needle is mated to each of the 2 tips of a syringe as shown in FIG. 11. This syringe having 2 hollow hypodermic needles is used to aspirate ascites fluid by puncturing the distended abdominal wall of a patient with tense ascites due to cancer or liver cirrhosis and then withdrawing fluid through the two needles at the same time.

### Example 7

### Use of a Multiple Needle Hemodialysis Catheter

After identifying the arteriorvenous (AV) access in the forearm of a patient in need of hemodialysis, a nurse makes preparations for conducting a hemodialysis procedure. The skin area of the patient is cleaned and sterilized with alcohol or other antiseptics. The nurse grips the wing of a hemodialysis needle device as illustrated in FIG. 17 inserts it into the venous side of the arteriovenous access as a draining needle. Another hemodialysis needle device (used as the infusing needle) is inserted to infuse the blood back into the patient after hemodialysis. The blood is drained out of the patient through the draining needle and the connecting catheter and is sent to the dialysis machine for filtering bodily wastes. The dialyzed blood is infused back into the patient through the infusing needle and the connecting catheter.

### Example 8

### Use of a Multiple Needle Hemodialysis Catheter

After identifying the arteriorvenous (AV) access in the forearm of a patient in need of hemodialysis, a nurse makes preparations for conducting a hemodialysis procedure. The skin area of the patient is cleaned and sterilized with alcohol or other antiseptics. The nurse grips the wing of a hemodialysis needle device as illustrated in FIGS. 19 and 20 and insert the needles of the device into the venous side of the arteriovenous access as a draining needle. The stylets are pulled back and removed from the needles after the needle device is inserted into the vein. Another hemodialysis needle device (used as the infusing needle) is inserted to infuse the blood back into the patient after hemodialysis, and after insertion the stylets are likewise pulled back and removed from the needles. The blood is drained out of the patient through the draining needle and the connecting catheter and is sent to the dialysis machine for filtering bodily wastes. The dialyzed blood is infused back into the patient through the infusing needle and the connecting catheter.

Although the present invention has been discussed in considerable detail with reference to certain preferred embodiments, other embodiments are possible. The steps disclosed for the present methods are not intended to be limiting nor are they intended to indicate that each step depicted is essential to the method, but instead are exemplary steps only. Therefore, the scope of the appended claims should not be limited to the description of preferred embodiments contained in this disclosure. All references cited herein are incorporated by reference to their entirety.

What is claimed is:

1. A lancet for use with a lancing device, the lancet having a plurality of needles mounted onto a lancet body, wherein the plurality of needles are mounted perpendicularly with respect to a direction in which the lancet is urged by the lancing device when the lancing device is actuated.

2. The lancet of claim 1, wherein each of the plurality of needles projects from the lancet body by an equal distance.

3. The lancet of claim 1, wherein the plurality of needles are at least 36 gauge needles.

4. A lancing system comprising the lancet of claim 1 and a lancing device capable of retaining the lancet and urging it forward in order to puncture a skin surface of a user when actuated.

5. The lancing system of claim 4, wherein one or more needles of the lancet projects from the surface of the lancet body by a different distance than one or more other needles of the lancet.

6. The lancing system of claim 5, wherein the lancing device comprises a depth adjustment control.

7. A method of obtaining a blood sample from an individual, comprising:
providing a lancet, the lancet comprising a lancet body and a plurality of needles mounted on the lancet body;
puncturing a skin surface of the individual with the plurality of needles; and
collecting blood from the skin surface of the individual, thereby obtaining the blood sample.

8. The method of claim 7, wherein at least one of the plurality of needles projects from the lancet body by a different distance compared to the remainder of the plurality of needles.

9. The method of claim 7, wherein the skin surface is located on a body part selected from the group consisting of a finger, a palm, a heel, an arm, a leg, and an earlobe.

10. The method of claim 7, wherein the lancet is delivered by a lancing device.

11. The method of claim 7, wherein the plurality of needles are solid.

12. The method of claim 7, wherein the plurality of needles are at least 34 gauge needles.

13. A needle device, comprising:
one or more needle hubs;
a plurality of needles attached to and in fluid communication with the one or more needle hubs, each of the needles being parallel to each other; and
a fluid conduit attached at a proximal end to the one or more needle hubs.

14. The needle device of claim 13, wherein the fluid conduit is a syringe.

15. The needle device of claim 14, wherein the syringe comprises a plurality of connecting tips, and wherein each of the connecting tips is connected to a needle hub.

16 The needle device of claims 13 or 14, wherein the one or more needle hubs comprise a plurality of needle seats for retaining a plurality of needles in each needle hub.

17. The needle device of claim 14, wherein the syringe comprises a plurality of chambers.

18. The needle device of claim 13, wherein the fluid conduit is a catheter.

19. The needle device of claim 18 wherein the catheter is attached at a distal end to a hemodialysis machine.

20. The needle device of claim 18, further comprising a stylet device having a plurality of stylets, wherein each of the stylets is adapted to fit within each of the plurality of needles.

21. The needle device of claim 18, wherein each of the plurality of needles comprises an opening on the side wall of the needle.
